(19) United States
(12) Patent Application Publication
(10) Pub. No.: US 2013/0304227 A1
(43) Pub. Date: Nov. 14, 2013

(54) METHOD OF FORMING LOCAL BONDS TO FASTEN A POROUS METAL MATERIAL TO A SUBSTRATE

(71) Applicant: ZIMMER, INC., Warsaw, IN (US)

(72) Inventor: Michael E. Hawkins, Columbia City, IN (US)

(73) Assignee: Zimmer, Inc., Warsaw, IN (US)

(21) Appl. No.: 13/864,483

(22) Filed: Apr. 17, 2013

Related U.S. Application Data
(60) Provisional application No. 61/646,602, filed on May 14, 2012.

(51) Int. Cl. A61F 2/30

(52) U.S. Cl. CPC A61F 2/30771
USPC 623/23.5; 219/137 R

(57) ABSTRACT

An orthopaedic prosthesis is provided having a porous layer and a substrate. A method is also provided for fastening the porous layer to the substrate. The porous layer defines a plurality of through-holes therein to accommodate localized bonding of the porous layer to the substrate through each of the plurality of through-holes.

### CLAIM OF PRIORITY

This application claims the benefit of U.S. Provisional Patent Application Ser. No. 61/646,602, filed on May 14, 2012, the benefit of priority is claimed hereby, and is incorporated by reference herein in its entirety.

### FIELD OF THE DISCLOSURE

The present disclosure relates to an orthopaedic prosthesis having a porous layer and a substrate, and to a method of fastening the porous layer to the substrate. More particularly, the present disclosure relates to a method of forming local bonds to fasten the porous layer to the substrate.

### BACKGROUND OF THE DISCLOSURE

Orthopaedic prostheses are commonly used to replace at least a portion of a patient's joint to restore or increase the use of the joint following traumatic injury or deterioration due to aging, illness, or disease, for example.

To enhance the fixation between an orthopaedic prosthesis and a patient's bone, the orthopaedic prosthesis may be provided with a porous metal layer. The porous metal layer may define at least a portion of the bone-contacting surface of the prosthesis to encourage bone growth and/or soft tissue growth into the prosthesis.

The porous metal layer may be metallurgically bonded to an underlying metal substrate. The metallurgical bond must be strong enough to withstand anatomical forces on the prosthesis when implanted. In certain embodiments, the metallurgical bond must meet or exceed the FDA-recommended bond strength of 2,900 psi. However, for various reasons, achieving a strong metallurgical bond may be difficult. First, pores in the porous metal layer create open spaces between the porous metal layer and the metal substrate, which may prevent complete surface contact between the porous metal layer and the metal substrate during the bonding process. Also, the porous metal layer and the substrate may be fabricated in complex shapes, which may prevent even surface contact between the porous metal layer and the metal substrate during the bonding process, even when pressure is applied to the porous metal layer and the metal substrate.

### SUMMARY

The present disclosure relates to an orthopaedic prosthesis having a porous layer and a substrate, and to a method of fastening the porous layer to the substrate. The porous layer defines a plurality of through-holes therein to accommodate localized bonding of the porous layer to the substrate through each of the plurality of through-holes.

According to an embodiment of the present disclosure, an orthopaedic prosthesis is provided including a substrate and a porous layer having a first surface that faces a patient's bone and a second surface that faces the substrate, the porous layer defining a plurality of through-holes that provide a direct pathway for an energy source from the first surface to the second surface.

According to another embodiment of the present disclosure, a method is provided for manufacturing an orthopaedic prosthesis. The method includes the steps of: providing a porous layer having a first surface that faces a patient's bone and a second surface, the porous layer defining a plurality of linear through-holes from the first surface to the second surface; placing the second surface of the porous layer against a substrate; and directing an energy source to the substrate through each of the plurality of through-holes to form local bonds between the porous layer and the substrate along the second surface of the porous layer.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a perspective view of a prosthetic distal femoral component of the present disclosure;

FIG. 2 is a cross-sectional view of the prosthetic distal femoral component of FIG. 1, taken along line 2-2 of FIG. 1, the prosthetic distal femoral component including a porous layer, a substrate, and an interlayer therebetween;

FIG. 2A is a detailed cross-sectional view of the circled area of FIG. 2;

FIG. 3 is a block diagram setting forth an exemplary method of the present disclosure;

FIG. 4 is a cross-sectional view similar to FIG. 2, and further showing an energy source forming local bonds between the porous layer and the interlayer; and

FIG. 4A is a detailed cross-sectional view of the circled area of FIG. 4.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

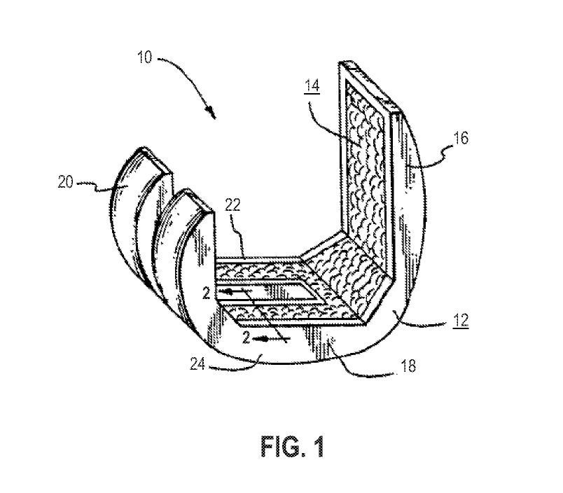

Referring initially to FIG. 1, an orthopaedic prosthesis is provided in the form of a prosthetic distal femoral component 10. While the orthopaedic prosthesis is illustratively a prosthetic distal femoral component 10, it is also within the scope of the present disclosure that the orthopaedic prosthesis may be in the form of a prosthetic proximal femoral component (e.g., a hip stem), a prosthetic tibial component, a prosthetic acetabular component, a prosthetic humeral component, or any other orthopaedic prosthesis, for example.

Prosthetic distal femoral component 10 includes articulating surface 12 and bone-contacting surface 14. Articulating surface 12 of prosthetic distal femoral component 10 includes anterior articulating portion 16 that is configured to articulate with a patient's patella (not shown), distal articulating portion 18 that is configured to articulate with a patient's tibia (not shown), and a pair of posterior, proximally extending condyles 20. Bone-contacting surface 14 of prosthetic distal femoral component 10 faces inwardly to contact the prepared or resected distal end of the patient's femur (not shown).

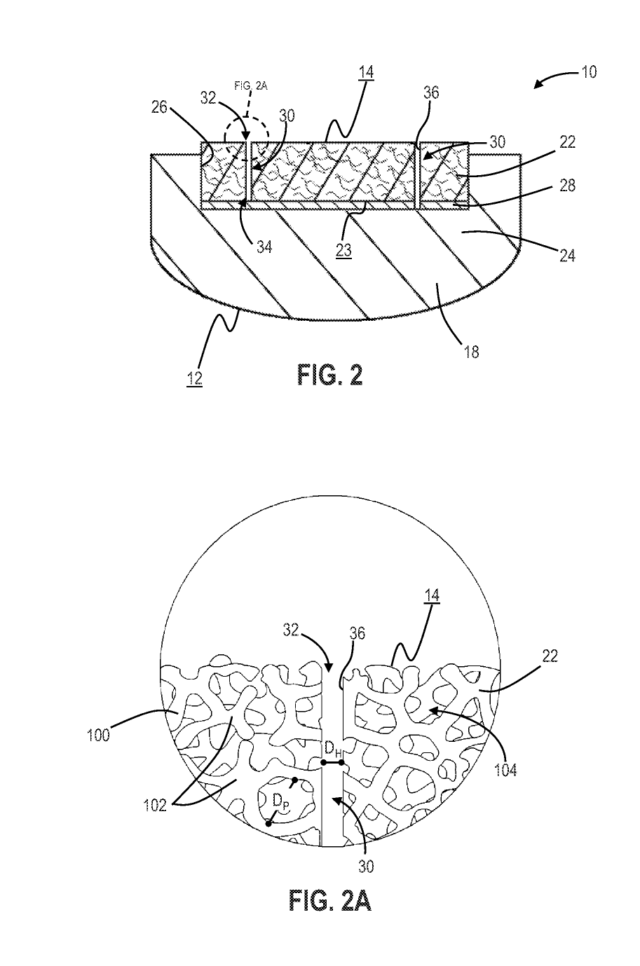

Referring next to FIG. 2, prosthetic distal femoral component 10 includes porous layer 22 coupled to substrate 24. Porous layer 22 may be disposed within recess 26 of substrate 24. Because porous layer 22 at least partially defines bone-contacting surface 14 of prosthetic distal femoral component 10, bone from the patient's femur may grow into porous layer 22 over time to enhance the fixation (i.e., osseointegration) between prosthetic distal femoral component 10 and the patient's femur. Porous layer 22 also includes a second, interfacing surface 23 opposite bone-contacting surface 14.

Porous layer 22 may be constructed of a highly porous biomaterial that is useful as a bone substitute and as cell and tissue receptive material. A highly porous biomaterial may have a porosity as low as 55%, 65%, or 75% or as high as 80%, 85%, or 90%.

An example of such a material is produced using Trabecular Metal™ Technology generally available from Zimmer, Inc., of Warsaw, Ind. Trabecular Metal™ is a trademark of Zimmer, Inc. Such a material may be a metal-coated scaffold that is formed from a reticulated vitreous carbon foam scaffold or substrate which is infiltrated and coated with a biocompatible metal, such as tantalum, by a chemical vapor deposition (“CVD”) process in the manner disclosed in detail in U.S. Pat. No. 5,282,861 to Kaplan, the entire disclosure of which is expressly incorporated herein by reference. In addition to tantalum, other metals such as niobium, or alloys of tantalum and niobium with one another or with other metals may also be used.

An exemplary porous tantalum material 100 is shown in FIG. 2A. Generally, the porous tantalum material 100 includes a large plurality of ligaments 102 defining open spaces or pores 104 therebetween, with each ligament 102 generally including a carbon core covered by a thin film of metal such as tantalum, for example. The open spaces 104 between the ligaments 102 form a matrix of continuous channels having no dead ends, such that growth of cancellous bone through the porous tantalum structure 100 is uninhibited. The porous tantalum structure 100 may include up to 75%, 85%, or more void space therein. Thus, porous tantalum structure 100 is a lightweight, strong porous structure which is substantially uniform and consistent in composition, and closely resembles the structure of natural cancellous bone, thereby providing a matrix into which cancellous bone may grow to provide fixation of prosthetic distal femoral component 10 to the patient's bone.

The porous tantalum structure 100 may be made in a variety of densities in order to selectively tailor the structure for particular applications. In particular, as discussed in the above-incorporated U.S. Pat. No. 5,282,861, the porous tantalum structure 100 may be fabricated to virtually any desired porosity and pore size, and can thus be matched with the surrounding natural bone in order to provide an improved matrix for bone ingrowth and mineralization.

Substrate 24 may be constructed of a biocompatible metal, such as cobalt or a cobalt chromium alloy. Substrate 24 may be cast or otherwise fabricated in a shape suitable of a particular orthopaedic application. The illustrative substrate 24 of FIG. 1, for example, is fabricated in a shape suitable for implantation on the patient's distal femur.

As shown in FIG. 2, porous layer 22 is indirectly coupled to substrate 24 via interlayer 28, which is positioned between porous layer 22 and substrate 24. Interlayer 28 may be constructed of a biocompatible metal that readily miscible with both the metal of porous layer 22 and the metal of substrate 24 for improved bonding. In embodiments where porous layer 22 is constructed of tantalum and substrate 24 is constructed of cobalt or a cobalt-chromium alloy, for example, interlayer 28 may be constructed of titanium, hafnium, manganese, niobium, palladium, zirconium, or an alloy thereof. In one embodiment, interlayer 28 is a pre-formed sheet of metal. In another embodiment, interlayer 28 is a surface coating that is deposited onto porous layer 22 and/or substrate 24. Interlayer 28 and a method of using interlayer 28 to form a diffusion bond is further described in U.S. Patent Application Publication No. 2009/0098310 to Hippensteel et al., the entire disclosure of which is expressly incorporated herein by reference.

Referring to FIGS. 2 and 2A, porous layer 22 includes a plurality of discrete through-holes 30 therein. According to an exemplary embodiment of the present disclosure, through-holes 30 are arranged in organized, staggered rows across porous layer 22. FIGS. 2 and 2A show through-holes 30 in distal portion 18 of prosthetic distal femoral component 10, but through-holes 30 may also be present in anterior portion 16 of prosthetic distal femoral component 10 and/or condyles 20 of prosthetic distal femoral component 10.

Each through-hole 30 extends entirely through porous layer 22, from first end 32 at the exposed bone-contacting surface 14 to second end 34 at interfacing surface 23. Optionally, through-hole 30 may continue extending beyond interfacing surface 23 of porous layer 22 and through interlayer 28 until reaching substrate 24 (as shown with respect to the right-most through-hole 30 of FIG. 2). Although the illustrative through-holes 30 are oriented perpendicularly relative to bone-contacting surface 14 and interfacing surface 23 of porous layer 22 in FIG. 2, it is also within the scope of the present disclosure that through-holes 30 may be angled within porous layer 22.

As shown in FIG. 2A, ligaments 102 of porous layer 22 cooperate to define wall 36 of through-hole 30. Due to the spaced-apart and varied arrangement of ligaments 102 in porous layer 22, wall 36 may be porous and jagged, as opposed to solid and smooth. Ligaments 102 terminate at or before reaching wall 36 of through-hole 30 and avoid extending into through-hole 30. In this manner, through-hole 30 provides substantially direct, linear, uninterrupted access through porous layer 22, without interference from ligaments 102 of porous layer 22. If through-hole 30 extends to interlayer 28, through-hole 30 may provide substantially direct, linear, uninterrupted access to interlayer 28 through porous layer 22. If through-hole 30 extends to substrate 24, through-hole 30 may provide substantially direct, linear, uninterrupted access to substrate 24 through porous layer 22.

Through-holes 30 may be pre-formed in porous layer 22. In one embodiment, through-holes 30 are formed in the reticulated vitreous carbon foam substrate before the substrate is infiltrated and coated with metal. Because the vitreous carbon foam substrate is readily deformable, through-holes 30 may be formed by piercing the vitreous carbon foam substrate with a pin or by cutting the vitreous carbon foam substrate, for example. In another embodiment, through-holes 30 are formed after the vitreous carbon foam substrate is infiltrated and coated with metal, such as by drilling into the coated metal or otherwise machining the coated metal.

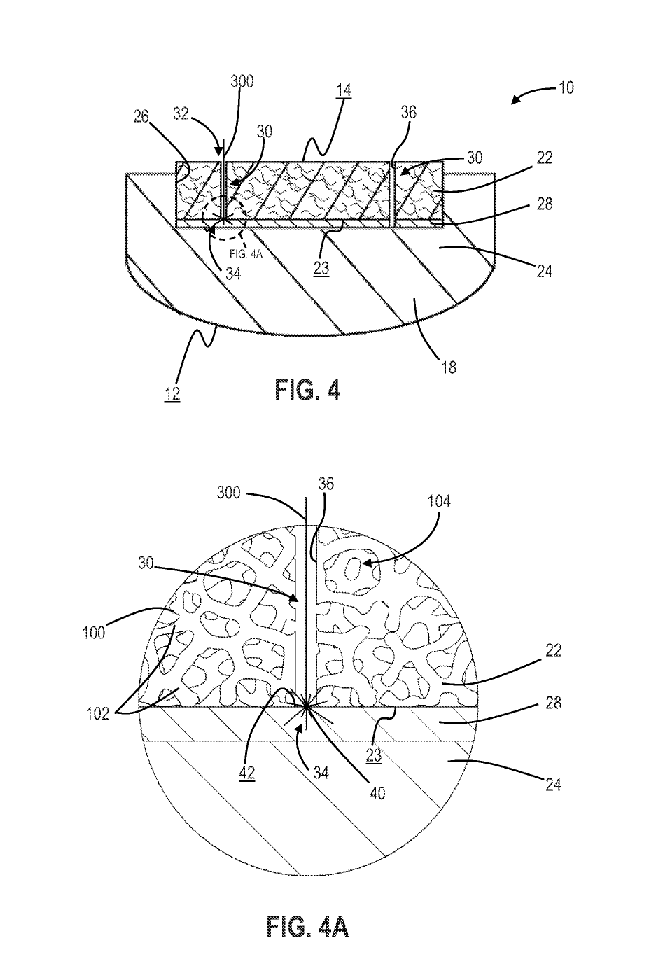

As shown in FIG. 2A, each through-hole 30 has a diameter D_{H}, which may be exaggerated in the drawings for purposes of illustration. The diameter D_{H} of each through-hole 30 is large enough to provide an energy source 300 (FIG. 4) with a substantially direct, linear, uninterrupted pathway through porous layer 22. However, the diameter D_{H} of each through-hole 30 may be minimized to maximize the presence of the surrounding porous layer 22 for bone ingrowth and stability. The diameter D_{H} of each through-hole 30 may be as small as 0.001″ (51 μm), 0.003″ (76 μm), or 0.005″ (127 μm), and as large as 0.007″ (178 μm), 0.009″ (229 μm), 0.011″ (279 μm), or more, for example.

According to an exemplary embodiment of the present disclosure, bone-contacting surface 14 of porous layer 22 is generally consistent in appearance, despite the presence of through-holes 30 in porous layer 22. To the naked eye, first end 32 of each through-hole 30 may look like an exposed pore 104 along bone-contacting surface 14 of porous layer 22. To achieve this result, the diameter D_{H} of each through-hole 30 may be about the same as, or smaller than, the average diameter D_{P} of pores 104. If the average diameter D_{P} of pores 104 in porous layer 22 is about 0.016″ (400 μm), 0.020″ (500 μm), or 0.024″ (600 μm), for example, the diameter D_{H} of each through-hole 30 may be less than 0.012″ (300 μm), 0.008″ (200 μm), or 0.004″ (100 μm).

Although through-holes 30 may look like pores 104 along bone-contacting surface 14 of porous layer 22, through-holes 30 differ from pores 104 beneath bone-contacting surface 14 of porous layer 22. An energy source 300 (FIG. 4) traveling along through-hole 30 may follow a substantially direct, linear, uninterrupted path through porous layer 22. By contrast, an energy source traveling through a pore 104 would eventually encounter an adjacent ligament 102. Thus, pores 104 do not provide direct, linear, uninterrupted access through porous layer 22.

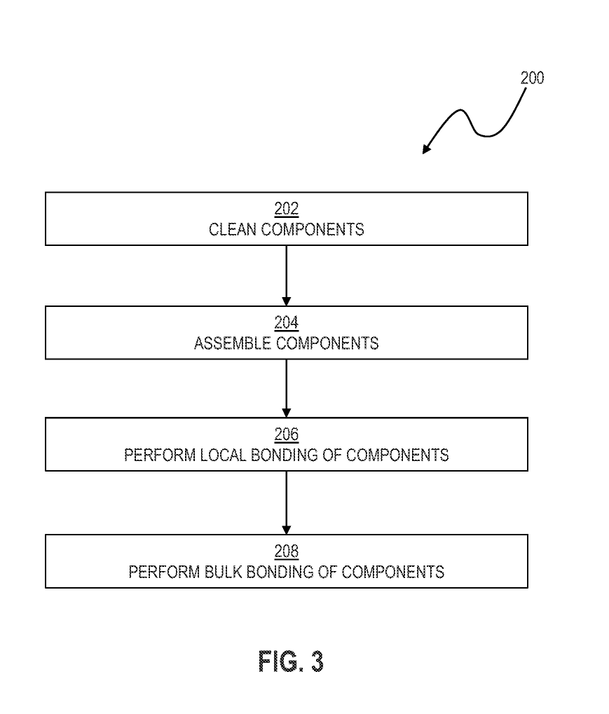

Referring next to FIG. 3, an exemplary method 200 is provided for manufacturing prosthetic distal femoral component 10.

First, in step 202 of method 200, the surfaces of porous layer 22, substrate 24, and/or interlayer 28 are cleaned. With respect to porous layer 22, for example, the interfacing surface 23 that will be bonded to interlayer 28 may be cleaned during the cleaning step 202. The cleaning step 202 may avoid corrosion and may improve subsequent bonding.

Next, in step 204 of method 200, porous layer 22, substrate 24, and interlayer 28 are assembled, as shown in FIG. 2. Some of the components may be pre-assembled before the assembling step 204. For example, if interlayer 28 is in the form of a metal sheet, interlayer 28 may be pre-attached or pre-bonded to substrate 24 before the assembling step 204. As another example, if interlayer is in the form of a surface coating, interlayer 28 may be pre-applied to substrate 24 before the assembling step 204.

Then, in step 206 of method 200, porous layer 22 is locally bonded to interlayer 28 and/or substrate 24. The local bonding step 206 may involve directing an energy source 300 through porous layer 22 via each through-hole 30, as shown in FIGS. 4 and 4A. More specifically, the local bonding step 206 may involve directing an energy source 300 from first end 32 to second end 34 of each through-hole 30.

A controller may be provided to automatically register energy source 300 to each through-hole 30. If the controller knows the orientation and spacing (e.g., staggered rows) of through-holes 30, the controller may automatically advance energy source 300 from one through-hole 30 to the next. Magnification and/or back-lighting may also be provided to properly register energy source 300 to each through-hole 30.

Upon reaching second end 34 of through-hole 30 through the substantially uninterrupted pathway, the energy source 300 impacts material at a localized point 40, as shown in FIGS. 4 and 4A. The energy from the energy source 300 is then converted into heat. The heat may be sufficient to cause localized softening and/or melting of the material at and around point 40 which intersects or is generally in line with the substantially uninterrupted pathway. In the illustrated embodiment of FIG. 4A, for example, the energy source 300 impacts interlayer 28 at point 40, and the heat may be sufficient to cause localized softening and/or melting of interlayer 28 along interface 42 (i.e., the surface of interlayer 28 that interfaces with second end 34 of through-hole 30) at and around point 40. If substrate 24 interfaces with second end 34 of through-hole 30 (as shown with respect to the right-most through-hole 30 of FIG. 4), the heat generated may be sufficient to cause localized softening and/or melting of substrate 24 where substrate 24 interfaces with second end 34 of through-hole 30. However, the heat may avoid causing localized softening and/or melting of ligaments 102 in porous layer 22, thereby maintaining the shape and structural integrity of porous layer 22. If, for example, porous layer 22 is constructed of tantalum (which has a melting point above 3,000° C.), substrate 24 is constructed of cobalt (which has a melting point around 1,495° C.) and interlayer 28 is constructed of titanium (which has a melting point around 1,650° C.), the heat may melt the relatively temperature-sensitive substrate 24 and/or interlayer 28 without melting the relatively temperature-stable porous layer 22.

The softened and/or molten material that forms along interface 42 may then interact with the surrounding ligaments 102 of porous layer 22. For example, the softened and/or molten material may spread out across interface 42 and interdigitate into the surrounding pores 104 of porous layer 22. Because the softened and/or molten material may be localized along interface 42 at and around point 40, the bulk properties of interlayer 28 and/or substrate 24 may remain unchanged. As the softened and/or molten material cools and re-hardens, localized metallurgical bonding may occur along interface 42.

A variety of different energy sources 300 may be used for the local bonding step 206. For example, the energy source 300 may be in the form of a laser beam, an electron beam, or a charged electrode. Also, the energy may be delivered from the energy source 300 continuously or in discrete pulses.

During the local bonding step 206, porous layer 22, substrate 24, and interlayer 28 may be subjected to an external clamping pressure to ensure good surface contact therebetween. Also, the local bonding step 206 may be performed in a controlled atmosphere, such as in a vacuum environment or in the presence of an inert gas, to minimize the presence of contaminants in the local bonds.

Depending on the number, strength, and location of the local bonds formed during the local bonding step 206, porous layer 22, substrate 24, and interlayer 28 of prosthetic distal femoral component 10 may be ready for implanting on the patient's distal femur after the local bonding step 206. The local bonding step 206 may produce the FDA-recommended bond strength of 2,900 psi between porous layer 22, substrate 24, and interlayer 28, for example.

Alternatively, an additional bulk bonding step 208 may be performed after the local bonding step 206. The bulk bonding step 208 may be required to achieve the FDA-recommended bond strength of 2,900 psi between porous layer 22, substrate 24, and interlayer 28, for example. During the subsequent bulk bonding step 208, the local bonds from the prior local bonding step 206 may hold porous layer 22, substrate 24, and/or interlayer 28 together to ensure good surface contact therebetween. The local bonds may supplement or enhance any external clamping pressure that is applied during the bulk bonding step 208. Additionally, the local bonds from the prior local bonding step 206 may ensure proper alignment between porous layer 22, substrate 24, and/or interlayer 28 during the subsequent bulk bonding step 208. In this manner, the local bonds may behave like tacks or pins in the prosthetic distal femoral component 10, holding together and aligning the complexly-shaped anterior portion 16, distal portion 18, and/or condyles 20 of the prosthetic distal femoral component 10.

The bulk bonding step 208 may involve a solid-state diffusion bonding process, which subjects the components to elevated temperatures and pressures. To maintain the structural integrity of porous layer 22, substrate 24, and interlayer 28, the pressure applied during the bulk bonding step 208 should be less than the compressive yield strength of porous layer 22, substrate 24, and interlayer 28. If porous layer 22 has the lowest compressive yield strength of 5,800 psi, for example, the applied pressure may be as low as 100 psi, 300 psi, or 500 psi and as high as 1,000 psi, 1,300 psi, or 1,500 psi. Also, the elevated temperature reached during the bulk bonding step 208 should be less than the melting point of porous layer 22, substrate 24, and interlayer 28. If substrate 24 and interlayer 28 have the lowest melting points of around 1,500° C., for example, the elevated temperature may be as low as 500° C., 600° C., or 700° C. and as high as 800° C., 900° C., or 1000° C. An exemplary diffusion bonding process is described in U.S. Pat. No. 7,686,203 to Rauguth et al., the entire disclosure of which is expressly incorporated by reference herein.

The bulk bonding step 208 may be performed in a controlled atmosphere, such as in a vacuum furnace or an inert furnace, to minimize the presence of contaminants in the bulk bonds.

After the bulk bonding step 208, prosthetic distal femoral component 10 may be ready for implanting on the patient's distal femur. For example, the bulk bonding step 208 may produce the FDA-recommended bond strength of 2,900 psi between porous layer 22, substrate 24, and interlayer 28.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An orthopaedic prosthesis comprising:
a substrate; and
a porous layer having a first surface for facing a patient's bone and a second surface that faces the substrate, the porous layer defining a plurality of through-holes that provide a direct pathway for an energy source from the first surface to the second surface.

2. The orthopaedic prosthesis of claim 1, wherein the porous layer completely surrounds each through-hole.

3. A method of manufacturing an orthopaedic prosthesis comprising the steps of:
providing a porous layer having a first surface for facing a patient's bone and a second surface, the porous layer defining a plurality of linear through-holes from the first surface to the second surface;
placing the second surface of the porous layer against a substrate; and
directing an energy source to the substrate through each of the plurality of through-holes to form local bonds between the porous layer and the substrate along the second surface of the porous layer.

4. The method of claim 3, further comprising the step of diffusion bonding the porous layer to the substrate after the directing step.

5. The method of claim 3, wherein the substrate comprises an interlayer between the porous layer and a second substrate.

6. The orthopaedic prosthesis of claim 2, wherein the plurality of through-holes have porous walls.

7. The orthopaedic prosthesis of claim 1, wherein the plurality of through-holes are arranged in rows.

8. The orthopaedic prosthesis of claim 1 further comprising bonding between the substrate and the second surface of the porous layer.

9. The orthopaedic prosthesis of claim 8, wherein said bonding includes a plurality of local bonds which each correspond to one of said plurality of through-holes.

10. The orthopaedic prosthesis of claim 1, wherein the substrate is positioned between the porous layer and a second substrate.

11. The orthopaedic prosthesis of claim 10, wherein said substrate defines a plurality of through-holes which are each situated in line with the direct pathway of one of said plurality of through-holes of the porous layer.

12. The method of claim 3, wherein said directing causes softened material of the substrate to interdigitate into pores of the porous layer.

13. The method of claim 12, wherein the porous layer is a porous metal layer that is receptive to tissue ingrowth.

14. An orthopaedic prosthesis, comprising:
a substrate;
a porous metal layer that is receptive to tissue ingrowth, the porous metal layer having a first surface for facing a patient's bone and a second surface that faces the substrate, the porous layer defining a plurality of through-holes that provide a direct pathway for an energy source from the first surface to the second surface; and
a plurality of local bonds spaced from one another along the substrate and effective to bond the substrate to the second surface of the porous metal layer, wherein the plurality of local bonds are each situated in line with the direct pathway of one of said plurality of through-holes.

15. The orthopaedic prosthesis of claim 14, wherein the plurality of local bonds are arranged in rows along the substrate.

16. The orthopaedic prosthesis of claim 14, wherein the plurality of local bonds provide bonding material that has interdigitated into pores of the porous metal layer.

17. The orthopaedic prosthesis of claim 14, wherein the plurality of through-holes have porous walls.
